(19) United States
(12) Patent Application Publication
(10) Pub. No.: US 2026/0114792 A1
(43) Pub. Date: Apr. 30, 2026

(54) Traumatic Brain Injury Sensor

(71) Applicants: Mackinnley Hammill, Townsend, MA (US); Callum Hammill, Townsend, MA (US)

(72) Inventors: Mackinnley Hammill, Townsend, MA (US); Callum Hammill, Townsend, MA (US)

(21) Appl. No.: 18/925,243

(22) Filed: Oct. 24, 2024

(51) Int. Cl. A61B 5/00

(52) U.S. Cl. CPC A61B 5/4064; A61B 5/6803; A61B 5/742; A61B 2562/0247

(57) ABSTRACT

A system for monitoring if a person has been exposed to a shock wave, pressure wave, or the like, which has a magnitude great enough to cause, or potentially cause injury, such as traumatic brain injury. The system may utilize a rupture disc which is formed so as to rupture upon exposure to pressure waves known to cause injury.

### BACKGROUND OF THE INVENTION

Traumatic brain injury (“TBI”) can be caused in several ways-including concussions or a series of concussions, head injuries from accidents, falls, sports injuries, and the like. Another cause of TBI is from pressure/shock waves which apply forces to the head that may in turn damage the brains of those exposed. Such pressure waves may arise from work sites such as construction sites, blast sites, demolition sites and the like. In other cases, soldiers in combat and/or training may be exposed to pressure or shock waves, often in a repeated fashion. Repeated exposure to explosion shockwaves by combat troops causes TBI. In some cases, TBI may be caused by repeated pressure waves even at relatively low levels, including by a soldier's own weapon. Battle fields vary in the way shockwaves bounce and reflect off surfaces, making it difficult to predict in all situations how dangerous the shockwaves may be.

The symptoms of TBI are common in 11-25% of veterans. This results in veterans having a poorer quality of life on average compared to the rest of the population. This leads to an overburdened healthcare system treating with injuries and treatments that could be easily prevented and avoided with monitoring to limit exposure of conditions that may cause TBI. According to one estimate, 185,000 veterans who use VA healthcare have mild TBI symptoms. When combined with post-traumatic stress disorder (“PTSD”), veterans with TBI can incur treatment costs that are much higher than their PTSD-only counterparts. In many cases, TBI symptoms are often confused with PTSD, leading to more difficult diagnoses.

TBI can lead to a variety of serious and lasting effects. For example, it can lead to cognitive impairments such as difficulties with memory, attention, problem-solving, and decision-making. TBI may also result in emotional changes, with many individuals experiencing mood swings, anxiety, depression, or irritability. In the worst cases, TBI may lead to violent outbursts such as mass shootings and/or suicide. TBI has been shown to cause adverse physical symptoms as well. For example, headaches, dizziness, fatigue, and issues with balance or coordination. Further, longer term impacts include social challenges, long term neurodegenerative diseases, and other quality of life problems.

It has been well established that preventative measures are the best option for avoidance of TBI, because currently, treatments after the fact are quite limited. Therefore, what is needed is an effective and simple device able to identify, visually or otherwise, when a wearer or user has been exposed to a pressure wave sufficient to cause TBI.

### SUMMARY OF THE INVENTION

The present disclosure relates generally to sensors operable to provide a visual or other indication of a pressure wave that may cause a traumatic brain injury. More particularly, the present disclosure relates to a rupture disc which may indicate exposure to a pressure wave sufficient to cause injury, particularly head injury.

The subject matter of this application may involve, in some cases, interrelated products, alternative solutions to a particular problem, and/or a plurality of different uses of a single system or article.

In one aspect, a rupture disc system is provided. The rupture disc system has a rupture disc holder which is formed of a base defining an internal space, a top opening, a bottom opening, and the rupture disc holder having a connection structure for connection to a helmet. Within the rupture disc holder is a rupture disc held within the internal space. The rupture disc is operable to rupture upon a pressure wave (or shock wave, and the like) impact of a predetermined pressure, and is exposed to the surrounding environment via an opening allowing air flow-such as a front opening covered by, e.g., a screen, or a vent or plurality of vents, among other options.

In another aspect, a helmet having a visual indicator of exposure to dangerous shock wave is provided. The helmet is formed having a body, and a strap. A rupture disc system is attached to an outside of the helmet body or to the strap. The rupture disc system comprises a rupture disc holder which is formed of a base defining an internal space, a top opening, a bottom opening, and the rupture disc holder having a connection structure for connection to a helmet. Within the rupture disc holder is a rupture disc held within the internal space. The rupture disc is operable to rupture upon a pressure wave impact of a predetermined pressure.

In yet another aspect, a method of measuring shock waves or pressure waves that may cause a traumatic brain injury to a person is provided. The method involves attaching a rupture disc system to a helmet. This rupture disc system comprises a rupture disc holder comprising a base defining an internal space, and a rupture disc held within the internal space of the rupture disc holder. The attaching of the rupture disc system may involve attaching the rupture disc holder to an outside of a helmet or a strap of the helmet, such that the rupture disc therein is exposed to and facing incoming pressure/shock waves. Once attached, upon impact of a notable wave or concussive force, and/or in general use, the wearer and/or others around the wearer are able to observe if the rupture disc has ruptured. If ruptured, the method further involves recording the rupture state in a database. The recording may include recording a date of rupture, location, and wearer identification.

### BRIEF DESCRIPTION OF DRAWINGS

FIG. 1 provides an exploded view of one embodiment of the present disclosure.

FIG. 2 provides an exploded view of a portion of an embodiment of the present disclosure.

FIG. 3 provides an exploded side view of yet another embodiment of the present disclosure.

FIG. 4 provides a view of still another embodiment of the present disclosure in use on a helmet.

FIG. 5 provides a side view of one embodiment of the present disclosure.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns a rupture disc operable to rupture and provide a visual indication at a pressure limit understood to cause or lead to traumatic brain injury when a person's head is exposed to such a pressure wave. The system involves a low cost, small, lightweight and mechanical solution to indicate exposure to potentially problematic pressure/shock waves. Rupture discs by nature are designed to fail when the maximum bending stress is equal to or greater than the material yield strength. Materials for the rupture disc may be, among other options, aluminum, graphite, Polytetrafluoroethylene (PTFE), and the like, as well as other known materials in the art.

In typical embodiments, the system includes a small rupture disc and rupture disc holder that is attachable to a helmet, such as the side/chin straps of the helmet, or on the outside surface of the helmet. Importantly, the system is generally preferred to be connected to the helmet or strap, or otherwise on the head area of the wearer to track pressure waves at the head, rather that elsewhere on the body. Given the non-uniformity of many pressure waves, a dangerous pressure wave may impact, e.g. the legs of an individual without being dangerous to the head, and vice versa. It is also important that the rupture disc and rupture disc holder be positioned on the outside of the helmet or otherwise be outward facing, because this is where the actual pressure waves impact. Sensors on, for example, the inside of a helmet are of little use because they are shielded from the bulk of the exposure due to limited airflow and in many cases being indirectly facing the impact direction. In other embodiments, it may be possible for the rupture disc holder to attach to the body of a wearer and/or an article of clothing of the wearer.

The rupture disc and disc holder are structured and formed so as to provide a visual indication that is easily identifiable when the rupture disc has been ruptured by a pressure wave, allowing for others around the wearer to see of the potential injury. In a particular, non-limiting embodiment, the rupture disc may be formed to rupture at the lower limit of the US military injury threshold of roughly 2.5-5 pounds per square inch (“psi”). This would signal when a soldier has been exposed to blasts that could cause TBIs. This level is lower than typical rupture discs and therefore structurally and functionally different from the prior art. The rupture disc may have varying shapes and is not limited to a traditional disc shape. The term “disc” is used herein to refer generally to the structure operable to rupture or otherwise physically permanently deform in a measurable manner upon exposure to a predetermined pressure or shock, and need not be circular in cross-sectional shape. In some embodiments, the rupture disc is directional, such that it protrudes outward in one direction-and the protruding direction is intended to face the pressure wave impact. In other embodiments, the rupture disc may be a flat membrane that does not have a directional mode of operation-and can register pressure exceeding the predetermined threshold by rupturing based on a direction of exposure in both directions.

In further embodiments, the rupture disc holder may be labeled or marked with a serial number and its activity may be tracked, namely user identification, location, date or dates of use, and rupture status (ruptured or not ruptured-indicating exposure or non exposure to dangerous pressure). In a further embodiment, the rupture disc holder may be equipped with a chip, RFID tag, or the like, for automatic scanning, tracking, and data logging. In still a further embodiment, the rupture disc holder may have a transmitter or transceiver which may remotely transmit information including status of the rupture disc to a remote location such as a database. In tracking embodiments, repeated, or even single rupture events indicating potential injury may be flagged for soldiers (or other operators such as demolition workers), and corrective or preventative action may be taken.

Preferably, the rupture disc system which includes the rupture disc and rupture disc holder contemplated herein allows for easy visual identification that the rupture disc has ruptured. One such embodiment utilizes a rupture disc indicator, which are used in other rupture disc applications such as industrial plants. For the present disclosure, preferably the indicator is small, low cost, and visually clear, but of course any visual rupture disc indicator may be used without straying from the scope of this disclosure. In one embodiment, the rupture disc indicator may utilize a colored wall or surface within the rupture disc, such as red, which is visible through the ruptured rupture disc. In some embodiments, a liquid dye may be positioned to release and, upon rupture or other actuation of the rupture disc, be released to color the surface of the rupture disc or surrounding material of the rupture disc holder. In other embodiments, a solid or powder dye may be positioned to release and, upon rupture or other actuation of the rupture disc, be released to color the surface of the rupture disc or surrounding material of the rupture disc holder, or otherwise be visually identifiable in the rupture disc holder. Other electronic rupture disc indicators, such as a light which turns on when the rupture disc is ruptured/actuated, or an audible alarm from a speaker or buzzer, and the like, may also be used.

The rupture disc holder may connect to the helmet or helmet strap in any manner. Typical connection structures include, but are not limited to: an adhesive, a clip, clamp, a slot, a slot through which a helmet strap may pass, a buckle on the strap, Velcro® or other hook and loop connection, a magnetic connection, a threaded connection, a twist on/off connection, snap on/off connection, and the like. In typical applications, connection to the helmet strap has been found to be optimal because of the ease of connection, ease of visual inspection by others, and limited interference with the helmet.

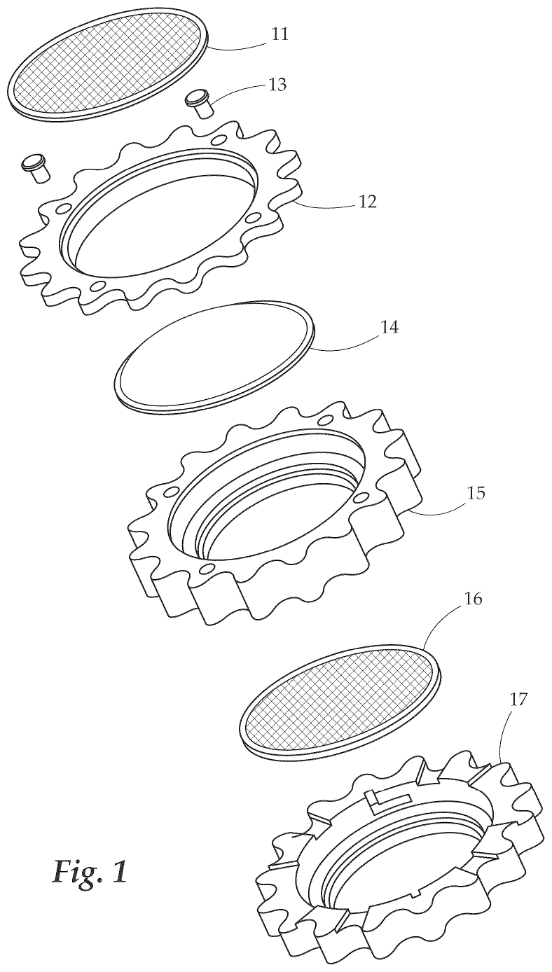

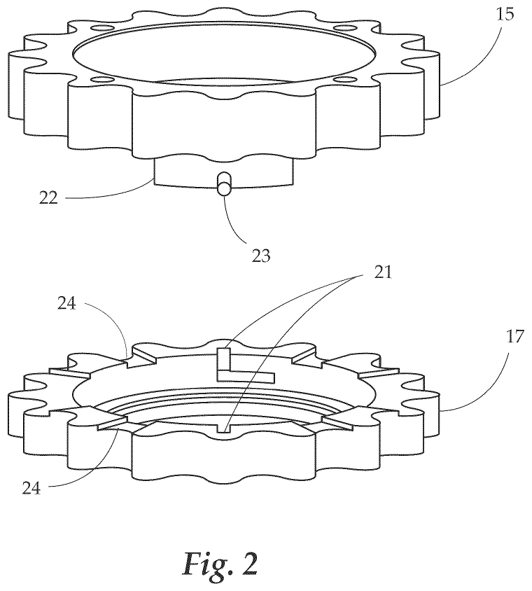

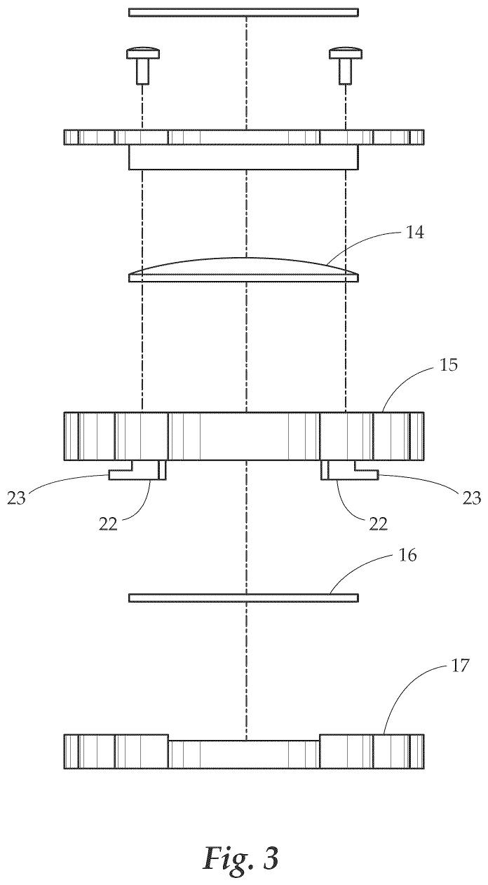

Turning now to FIG. 1, an embodiment of the rupture disc system is provided in an exploded view. The rupture disc 14 fits into and is held within the rupture disc holder. The rupture disc holder includes a base 15 which defines a ridge or shoulder into which the rupture disc 14 removably seats. Spent rupture discs can be quickly replaced with new ones by opening the rupture disc holder, removing the spent rupture disc, and replacing it with a new rupture disc 14. The rupture disc holder further has a bottom 17 attached to the bottom of the base 15 and a top 12 attached to the top of the base 15. Each of top piece 12, base 15, and bottom piece 17 have a scalloped shape which has been unexpectedly found to allow for effective pressure wave exposure to the rupture disc 14, as well as easy handling, attachment to helmet, and assembly/disassembly. A screen 11 seats into the rupture disc holder top 12, and the top 12 attaches to base 15 in any manner, and in the embodiment shown, via threaded screws or friction fit pins. The screen 11 is used as a cover to protect the rupture disc, but allows for easy air flow to the rupture disc so as to not interfere with its operation. Similarly, the bottom 17 of the rupture disc holder holds screen 16 which also allows for air flow in and out, and prevents trapped air from interfering with the rupture disc 14 operation. The rupture disc 14 shown here has a convex shockwave facing side, shown here as facing to the top 12 of the rupture disc holder. Base 15 connects to bottom piece 17, in this embodiment, via a twist lock connection, as shown in FIGS. 2 and 3.

FIG. 2 shows another embodiment of the rupture disc holder of the present disclosure. In this view, base 15 connects to bottom piece 17 by a twist lock connection. A flange 22 extends downward from a lower surface of the base 15, and has a pin 23 extending outward. A similar or identical structure is present on the opposite side of the base 15 (not shown). The pin engages with the “L” shaped slots 21 on the bottom 17, and the base 15 can be pushed so the pin 23 passes through the vertical portion of the slot 21, and then twisted to secure the pin 23 into the horizontal portion of the slot 21. In some cases, a ramp (not shown) or angled horizontal portion of the slot 21 may be employed to cause the base 15 and bottom 17 to tighten against each other when twisted, but this is not required. Bottom part 17 also forms a series of vents 24. Vents 24 provide additional gas venting to prevent a negative impact in operation of the rupture disc 14. In other words, if the rupture disc holder is not able to easily vent air behind the rupture disc, the impact of a shockwave may not be adequately measured, because trapped air behind the rupture disc could prevent it from deforming (which requires displacement of the air behind it). Thus, a plurality of the large vents 24 serve to effectively vent air to allow for precise and adequate reading of the pressure-namely the pressure differential from one side of the rupture disc to the other.

FIG. 3 provides a side exploded view of an embodiment of the present disclosure. Rupture disc 14 seats into the base 15 via, e.g. a seat, shoulder, snap-fit connection of one or more ridges that the rupture disc 14 may seat between, and the like. Top 12 has a downwardly extending annular flange which fits into the top opening formed by the base 15, and is held to the base 15 by screws 13. Screens 11 and 16 cover a top and bottom of the central opening of the rupture disc holder, where the rupture disc 14 is held. The twist lock structure of flange 22 and pin 23 can be seen in this view as well, as discussed in detail above with respect to FIG. 2.

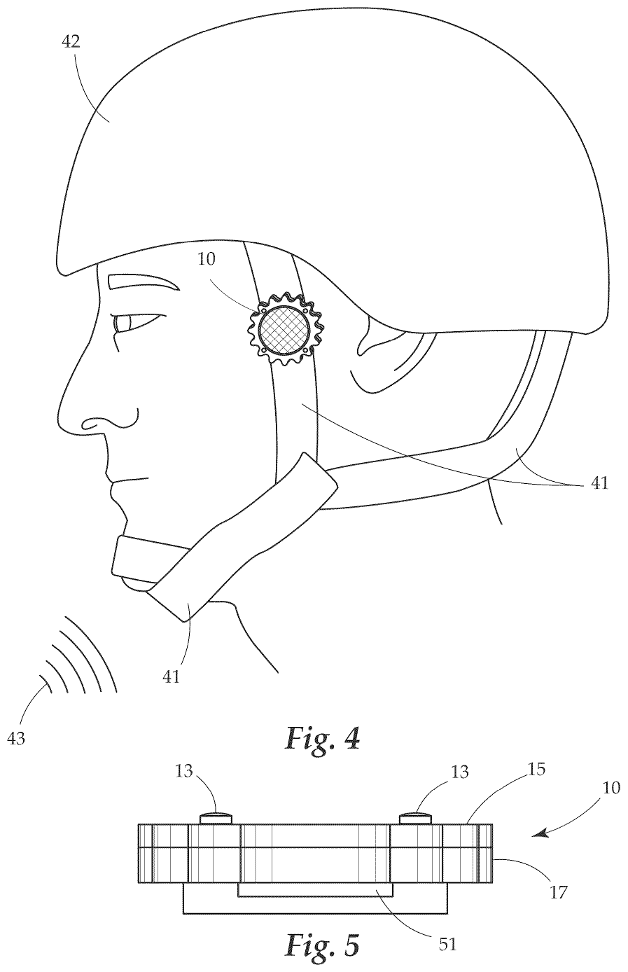

FIG. 4 shows a view of the rupture disc holder 10 with a rupture disc positioned therein attached to a helmet 42 and positioned on a user's head. It should be noted that any helmet and/or strap configuration may be used without straying from the scope of this disclosure. The rupture disc holder 10 is attached to strap 41 of helmet 42. The front opening faces outward and can identify if a shockwave 43 is of sufficient force to potentially cause injury, namely TBI. In use, the rupture disc may be easily replaceable when the rupture disc has ruptured, been actuated, or otherwise needs replacing due to age, getting dirty, and the like. In the embodiment shown, the base 15 may be twisted to release the twist lock from bottom 17, the rupture disc may be removed through the lower side of the base 15, and replaced, and then the base 15 may be reconnected to the bottom part 17. This allows the bottom part 17 to remain connected to the helmet strap or body.

FIG. 5 shows a view of an assembled embodiment of the present disclosure. In this embodiment, the rupture disc holder 10 only has a base 15 and bottom 17, without a top as shown in the embodiment of FIG. 1. Screws 13 hold screen (not shown) to base 15. The base 15 is removably connected to the bottom piece 17. A slot connector 51 allows the helmet strap 41 to be threaded through the slot 51 to connect the rupture disc holder 10 to the helmet. Of course, other connecting structures may be used without straying from the scope of this disclosure. For example, in another slot embodiment, there may be a thin central opening at the bottom of the slot connector 51, allowing the strap 41 material to fit in from the bottom and into the slot. This structure may prevent the need to fully remove the strap 41 from end connectors such as a clasp.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is

1. A rupture disc system comprising:
a rupture disc holder, the rupture disc holder comprising a base defining an internal space, a top opening, a bottom opening, and the rupture disc holder having a connection structure for connection to a helmet; and
a rupture disc held within the internal space of the rupture disc holder.

2. The rupture disc system of claim 1 wherein the rupture disc is formed to rupture upon exposure to a pressure between approximately 2.5-5 psi.

3. The rupture disc system of claim 1 further comprising a rupture indicator, the rupture indicator providing a visual representation when the rupture disc has ruptured.

4. The rupture disc system of claim 3 wherein the rupture disc indicator comprises a liquid dye released upon rupture of the rupture disc, a powder dye released upon rupture of the rupture disc, or a brightly colored surface behind the rupture disc that is visible when the rupture disc is ruptured.

5. The rupture disc system of claim 1 wherein the top opening and bottom opening comprise screens positioned over the internal space, ore are vents.

6. The rupture disc system of claim 1 wherein the rupture disc holder further comprises a bottom piece removably connected to the base, the connection structure being formed on the bottom piece.

7. The rupture disc system of claim 6 wherein the bottom piece is connected to the base by a twist lock connection.

8. The rupture disc system of claim 1 wherein the base defines a ridge or shoulder into which the rupture disc removably seats.

9. The rupture disc system of claim 1 wherein the connector structure comprises one of an adhesive, a clip, clamp, a slot, a slot through which a helmet strap may pass, a buckle, a hook and loop connection, a magnetic connection, a threaded connection, a twist on/off connection, and a snap on/off connection.

10. The rupture disc system of claim 6 wherein the rupture disc holder further comprises a top piece removably connected to the base, the top piece defining a central opening aligned with an opening of the internal space and allowing viewing of the rupture disc, and comprising a screen covering the central opening.

11. A helmet having a visual indicator of exposure to dangerous shock wave comprising:
the helmet having a body, and a strap;
a rupture disc system attached to an outside of the helmet body or the strap, the rupture disc system comprising:
a rupture disc holder, the rupture disc holder comprising a base defining an internal space, a top opening, a bottom opening, and the rupture disc holder having a connection structure connecting the rupture disc holder to the outside of the helmet body or the strap; and
a rupture disc held within the internal space of the rupture disc holder.

12. The rupture disc system of claim 11 wherein the rupture disc is formed to rupture upon exposure to a pressure between approximately 2.5-5 psi.

13. The rupture disc system of claim 11 further comprising a rupture indicator, the rupture indicator providing a visual representation when the rupture disc has ruptured.

14. The rupture disc system of claim 13 wherein the rupture disc indicator comprises a liquid dye released upon rupture of the rupture disc, a powder dye released upon rupture of the rupture disc, or a brightly colored surface behind the rupture disc that is visible when the rupture disc is ruptured.

15. The rupture disc system of claim 11 wherein the top opening and bottom opening comprise screens positioned over the internal space, ore are vents.

16. The rupture disc system of claim 11 wherein the rupture disc holder further comprises a bottom piece removably connected to the base, the connection structure being formed on the bottom piece.

17. The rupture disc system of claim 16 wherein the bottom piece is connected to the base by a twist lock connection, and wherein the base defines a ridge or shoulder into which the rupture disc removably seats.

18. The rupture disc system of claim 11 wherein the connector structure comprises one of an adhesive, a clip, clamp, a slot, a slot through which a helmet strap may pass, a buckle, a hook and loop connection, a magnetic connection, a threaded connection, a twist on/off connection, and a snap on/off connection.

19. The rupture disc system of claim 16 wherein the rupture disc holder further comprises a top piece removably connected to the base, the top piece defining a central opening aligned with an opening of the internal space and allowing viewing of the rupture disc, and comprising a screen covering the central opening.

20. A method of measuring shock waves or pressure waves that may cause a traumatic brain injury to a person comprising the steps of:
attaching a rupture disc system to a helmet, the rupture disc system comprising a rupture disc holder comprising a base defining an internal space; and
a rupture disc held within the internal space of the rupture disc holder;
wherein the attaching of the rupture disc system comprises attaching the rupture disc holder to an outside of a helmet or a strap of the helmet;
observing that the rupture disc has ruptured; and
recording the rupture state in a database, the recording comprising recording a date of rupture, location, and wearer identification.
